(19) United States
(12) Patent Application Publication
(10) Pub. No.: US 2025/0176922 A1
(43) Pub. Date: Jun. 5, 2025

(54) PATIENT EXAMINATION AUGMENTED REALITY (PEAR) SYSTEM

(71) Applicant: University of Central Florida Research Foundation, Inc., Orlando, FL (US)

(72) Inventors: Mindi Anderson, Orlando, FL (US); Desiree A. Diaz, Orlando, FL (US); Ryan McMahan, Orlando, FL (US); Steve Zelenty, Orlando, FL (US); Francisco Guido-Sanz, Orlando, FL (US)

(21) Appl. No.: 19/049,142

(22) Filed: Feb. 10, 2025

Related U.S. Application Data
(63) Continuation of application No. PCT/US2023/029968, filed on Aug. 10, 2023.
(60) Provisional application No. 63/369,814, filed on Jul. 29, 2022.

(51) Int. Cl. A61B 5/00; A61B 90/00; A61B 90/50; G06F 3/01; G06T 19/00; G16H 10/60

(52) U.S. Cl. CPC A61B 5/742; G06F 3/011; G06T 19/006; G16H 10/60; A61B 2090/365; A61B 2090/502; G06T 2210/41

(57) ABSTRACT

Described herein relates to a system and method for optimizing a physical patient examination through augmented reality (AR), virtual reality (VR), mixed reality (MR), and/or extended reality (XR) to augment the appearance of the physical patient representation and/or to simulate physical movements, aspects, and/or behaviors that the physical patient representation is not capable of on its own, while also affording the ability to provide physical contact with the physical patient representation. Additionally, these enhancements may offer several benefits in the healthcare domain, such as improved learning, training, mentoring, practice, and/or case planning. Furthermore, these enhancements may be useful for other domains, such as biology education, clothing retail, and any service or activity involving human representations.

### CROSS-REFERENCE TO RELATED APPLICATIONS

This nonprovisional application is a continuation of PCT International Patent Application No. PCT/US2023/029968 entitled “PATIENT “EXAMINATION AUGMENTED REALITY (PEAR) SYSTEM” with an international filing date of Aug. 10, 2023, by the same inventors, which claims the benefit of U.S. Provisional Application No. 63/396,814 entitled “PATIENT EXAMINATION AUGMENTED REALITY (PEAR) SYSTEM” filed Aug. 10, 2022, by the same inventors, all of which are incorporated herein by reference, in entirety, for all purposes.

### BACKGROUND OF THE INVENTION

### 1. Field of the Invention

This invention relates, generally, to patient examination. More specifically, it relates to a system and method for optimizing a physical patient examination through augmented reality (AR), virtual reality (VR), and/or mixed reality (MR) to augment the appearance of the physical patient representation and to simulate physical movements, aspects, and/or behaviors that the physical patient representation is not capable of on its own, allowing for realistic movement and/or gestures, a diversity of races/ethnicities and/or disorders while still providing physical contact.

### 2. Brief Description of the Prior Art

Manikins have been available for use in medicine as early as the 19^{th} Century. As such, they are often used for student learning, however, not without their limitations. Presently while manikins are available for student learning, they lack realism, limiting their applicability to real world experiences. This includes but is not limited to both movement skin tones, and/or skin disorders. As such, certain types of assessment, such as newborn neuromuscular reflexes, are not able to be elicited with the manikins currently known in the art.

Recent advances in manikins have enabled use of augmented reality, for example, the Optical See-Through Head-Mounted Display (hereinafter “OST-HMD”) approach. However, currently known patient examination using this technology would not be capable of making a Black physical patient representation appear as an Asian virtual patient representation due to the transparency artifacts of the optical combiners used to create the optical see-through approach. In other words, the user would perceive an Asian virtual patient overlayed on the Black physical patient representation.

Another type of currently known patient examination apparatuses involves the use of a patient bed system—such as the one disclosed in U.S. Pat. No. 9,679,500 (issued Jun. 13, 2017). This type of patient examination system works with any physical patient representation, from simple static manikin to complex patient representations, and can be easily transported to any new location with a physical patient representation by simply moving the Video See-Through Head-Mounted Display (hereinafter “VST-HMD”), which is already designed to be a portable system. Furthermore, unlike the body shell in the previously cited patent, the physical patient representations used for the current invention afford more-realistic physical touching, simulated grasping, and interactions, such as using a real measuring tape to measure the circumference of a physical manikin newborn's chest while simultaneously seeing the augmented infant over the physical one.

Accordingly, what is needed is a safe, efficient, easy-to-use, and accurate patient examination system which affords the ability to physically touch and grasp the virtual patient representation and characteristics. However, in view of the art considered as a whole at the time the present invention was made, it was not obvious to those of ordinary skill in the field of this invention how the shortcomings of the prior art could be overcome.

### SUMMARY OF THE INVENTION

The long-standing but heretofore unfulfilled need, stated above, is now met by a novel and non-obvious invention disclosed and claimed herein. In an aspect, the present disclosure pertains to a patient examination system for optimizing a physical patient examination. In an embodiment, the system may comprise the following: (a) an extended reality component communicatively coupled to at least one user-input actuator, the extended reality component configured to scan at least one portion of a physical patient representation to overlay a virtual patient representation on the physical patient representation, the user-input actuator configured to receive at least one stimulus from a user to at least one portion of the overlayed virtual patient representation; and (b) a computing device having at least one processor communicatively coupled to the extended reality component, the computing device configured to receive the scan of the at least one portion of the physical patient representation from the extended reality component. In this embodiment, the computing device may be communicatively coupled to a display device, the display device configured to visualize at least one portion of the virtual patient representation. In this manner, upon receiving the stimulus from the at least one user, the extended reality headset may be configured to generate a response within the overlayed virtual patient representation disposed upon at least one portion of the physical patient representation, such that the extended reality component may transmit the response to the display device.

In some embodiments, a memory of the computing device may comprise a deep-learning module comprising a plurality of trained appropriate responses and/or trained known responses. As such, when the at least one user provides a stimulus to the virtual, augmented, and/or mixed reality representation, the extended reality component may be configured to transmit a signal to the at least one processor, such that the virtual patient representation may convey the at least one trained appropriate response, and/or at least one trained known response based on the provided stimulus. Additionally, in these other embodiments, the deep leaning module may further comprise a plurality of trained movements and/or trained sounds.

In some embodiments, the at least one processor may be configured to alter at least one visual characteristic of the physical patient representation with at least one visual characteristic of the virtual patient representation within the display device associated with the computing device and/or the extended reality component. In some embodiments, the extended reality component may be selected from a group comprising: augmented reality, mixed reality, and/or virtual reality.

In some embodiments, the deep-learning module of the patient examination system may be communicatively coupled to at least one alternative computing device and/or at least one alternative display device. In these other embodiments, the at least one processor may be configured to display an interaction of the at least one user and the virtual patient representation on the at least one alternative computing device and/or at least one alternative display device, such that at least one alternative user may be able to view, in real-time, the interaction of the at least one user and the virtual patient representation. Additionally, in these other embodiments, the deep-learning module may comprise a plurality of trained background data sets and/or a plurality of trained health information data sets with respect to the virtual. As such, the at least one processor may be configured to overlay at least one of the plurality of trained background data sets and/or at least one of the plurality of trained health information data sets of the virtual patient representation with the view of an interaction of the at least one user and the virtual patient representation on the display device, simultaneously and in real-time.

In addition, in some embodiments, when the extended reality component overlays at least one portion of the physical patient representation with at least one associated portion of the virtual patient representation, the extended reality component may be configured to replace at least one aspect of the physical patient representation with at least one computer-generated aspect of the virtual patient representation within the display device associated with the computing device and/or the extended reality component.

Moreover, another aspect of the present disclosure pertains to a method for optimizing patient examination training. In an embodiment, the method may comprise the following steps: (a) scanning a physical patient representation disposed about an extended reality component, such that a virtual patient representation may be overlayed upon at least one portion of the scanned physical patient representation; (b) generating, via the extended reality component, a response associated with an inputted stimulus from at least one user onto at least one portion of the virtual patient representation, such that the stimulus may be inputted via at least one user-input actuator communicatively coupled with the extended reality component; (c) comparing, via a computing device having at least one processor communicatively coupled to the extended reality component, the associated response with a plurality of trained appropriate responses and/or trained known responses; and (d) transmitting, via the computing device, an examination score to a display device associated with the computing device and/or the extended reality component, such that the examination score may be calculated based on the comparison between the associated response and at least one response of the plurality of trained appropriate responses and/or trained known responses.

In some embodiments, a memory of the computing device comprises a deep-learning module comprising the plurality of trained appropriate responses and/or trained known responses. In these other embodiments, the method may further comprise the step of, transmitting, via the extended reality component, at least one signal to the at least one processor, such that the virtual patient representation may convey the at least one appropriate response and/or at least one known response based on the provided stimulus. In this manner, the deep leaning module may further comprise a plurality of trained movements and/or trained sounds.

In some embodiments, the method may further comprise the step of, altering, via the at least one user-input actuator, at least one visual characteristic of the physical patient representation with at least one visual characteristic of the virtual patient representation within the display device associated with the computing device and/or the extended reality component. In some embodiments, the extended reality component may be selected from a group comprising: augmented reality, mixed reality, and/or virtual reality.

In some embodiments, the deep-learning module of the patient examination system may be communicatively coupled to at least one alternative computing device and/or at least one alternative display device. In these embodiments, the method may further comprise the step of, displaying, via the at least one processor, an interaction of the at least one user and the virtual patient representation on the at least one alternative computing device and/or at least one alternative display device, such that at least one alternative user views, in real-time, the interaction of the at least one user and the virtual patient representation. In these other embodiments the deep-learning module may further comprise a plurality of trained background data sets and/or a plurality of trained health information data sets with respect to the virtual patient representation. In this manner, the method may comprise the step of, overlaying, via the at least one processor, at least one of the plurality of background data sets and/or at least one of the plurality of health information data sets of the virtual patient representation with the view of an interaction of the at least one user and the virtual patient representation on the display device, simultaneously and in real-time.

In some embodiments, the method may further comprise the step of, replacing at least one aspect of the physical patient representation with at least one computer-generated aspect of the virtual patient representation within the display device associated with the computing device and/or the extended reality component.

In some embodiments, the patient examination system may provide several benefits to patient simulation approaches. As compared to physical patient representations, the patient examination system may be configured to simulate any visual characteristic, such as jaundice and/or cyanosis, and/or visual behavior, such as rooting and palmar grasp reflexes. Additionally, as compared to other simulations that use optical see-through head-mounted displays (i.e., OST-HMDs) and systems (e.g., the Microsoft® HoloLens® 2) to augment physical patient representations, in these other embodiments, the patient examination system may be configured to completely supersede and/or replace the visual qualities of the physical patient representation.

Additionally, in some embodiments, the patient examination system may be configured to be used for education, training, mentoring, practice, case planning, assessment, evaluation, research, and/or any patient training method known in the art. Moreover, the patient examination system may also be used in other domains, such as outside of medical patient simulation. Accordingly, in these other embodiments, the patient examination system may be used to show the internal anatomy (e.g., organs, veins) of a physical representation (e.g., a static manikin and/or mannequin), which would likely facilitate anatomy education.

Additionally, in clothing retail, in some embodiments, the patient examination system may be configured to visualize different colors and/or patterns of clothing (e.g., a shirt) while being able to physically touch and/or feel the real fabric of the clothing on a manikin/manikin. In this manner, in these other embodiments, the patient examination system may be used in any domain in which the user may want to physically touch a representation of a patient and/or a human but also have the ability to dynamically change the visual characteristics of the human representation.

Furthermore, as compared to purely virtual patient simulations (e.g., VR simulations), in some embodiments, the patient examination system may be configured to provide the user the ability to physically touch and/or grasp the virtual patient representation, as the virtual patient representation may be co-located with the physical patient representation. As such, for example, when at least one user reaches out to touch the bottom of the virtual patient's foot, the hand of at least one user may come into contact with the bottom of the physical patient representation's foot at the same time that the at least one user sees their hand come into contact with the virtual foot. Accordingly, the passive haptic approach may afford a psychomotor perception and/or skills which may not be available in augmented reality, virtual reality, mixed reality and/or extended reality simulations, including but not limited to examining the patient's body by touch (e.g., palpating), as most simulations known in the art do not afford any force feedback and/or haptics, other than simple vibrotactile feedback (via the vibration motors in the at least one user-input actuator).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not restrictive.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A is a system diagram of patient evaluation and/or assessment using a patient examination system, according to an embodiment of the present disclosure.

FIG. 1B is a visual representation of an exemplary configuration of a patient examination system, according to an embodiment of the present disclosure.

FIG. 2 is a graphical representation of an exemplary configuration of a virtual model of a patient for a patient examination system, according to an embodiment of the present disclosure.

FIG. 3 is a visual representation of an exemplary configuration of a virtual patient representation overlayed on a physical patient representation (e.g., a manikin) of a patient examination system, according to an embodiment of the present disclosure.

FIG. 4 is a graphical representation of an alternative virtual patient representation of a patient examination system, according to an embodiment of the present disclosure.

FIG. 5A is a visual representation of an exemplary configuration of a physical patient representation of a patient examination system, according to an embodiment of the present disclosure.

FIG. 5B is a visual representation of an exemplary configuration of dynamic virtual scaling and/or alignment of a physical patient representation of a patient examination system, according to an embodiment of the present disclosure.

FIG. 5C is a visual representation of an exemplary configuration of a virtual patient representation overlaid on a physical patient representation based on dynamic scaling and/or alignment of the physical patient representation of a patient examination system, according to an embodiment of the present disclosure.

FIG. 6 is a visual representation of an exemplary configuration of at least one user interacting with a virtual patient representation within an Augmented Reality component of a display device (e.g., a head-mounted display) associated with a patient examination system, according to an embodiment of the present disclosure.

FIG. 7A is a visual representation of an exemplary configuration of an interaction between at least one user and at least one virtual patient of a patient examination system, according to an embodiment of the present disclosure.

FIG. 7B is a visual representation of an exemplary configuration of an initial reflex response of at least one virtual patient based on the interaction of at least one user and the at least one virtual patient of a patient examination system, according to an embodiment of the present disclosure.

FIG. 7C is a visual representation of an exemplary configuration of a completion of a reflex response based on an interaction of at least one user and at least one virtual patient of a patient examination system, according to an embodiment of the present disclosure.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention. Elements/components shown in diagrams are illustrative of exemplary embodiments of the disclosure and are meant to avoid obscuring the disclosure. Any headings, used herein, are for organizational purposes only and shall not be used to limit the scope of the description or the claims. Furthermore, the use of certain terms in various places in the specification, described herein, are for illustration and should not be construed as limiting.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

### Definitions

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

In the following description, for the purposes of explanation, numerous specific details are set forth in order to provide a thorough understanding of embodiments of the present technology. The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program PIN embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program PIN embodied on a computer readable medium may be transmitted using any appropriate medium, including but not limited to wireless, wire-line, optical fiber cable, radio frequency, etc., or any suitable combination of the foregoing. Computer program PIN for carrying out operations for aspects of the present invention may be written in any combination of one or more programming languages, including an object-oriented programming language such as Java, C#, C++, Python, Swift, MATLAB, and/or the like and conventional procedural programming languages, such as the “C” programming language or similar programming languages.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computing device, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

As used herein, the term “Application Programming Interface” (hereinafter “API”) refers to any programming or software intermediary that allows an application to communicate with a third-party application. For ease of reference, the exemplary embodiment, described herein, refers to a programming which communicates with hotel, airfare, golf, spa, and rental car applications, but this description should not be interpreted as exclusionary of other types of third-party applications.

As used herein, the term “computing device” refers to any functional electrical component known in the art which can perform substantial computations, including numerous arithmetic operations and/or logic operations without human intervention. Non-limiting examples of the computing device may comprise a laptop, a mobile device, a computer, and/or a tablet. For ease of reference, the exemplary embodiment described herein refers to a mobile device and/or a computer, but this description should not be interpreted as exclusionary of other functional electrical components.

As used herein, the term “display device” refers to any functional electrical component known in the art which can present any aspect known in the art information in visual and/or tactile form without human intervention. Non-limiting examples of the display device may comprise a television, a graphical user-interface, a head-mounted display, X-Ray Scans, CT-Scans, MRIs, e-book, LCD display, LED display, and/or CRTs. For ease of reference, the exemplary embodiment described herein refers to a head-mounted display, but this description should not be interpreted as exclusionary of other functional display components.

As used herein, the term “communicatively coupled” refers to any coupling mechanism configured to exchange information (e.g., at least one electrical signal) using methods and devices known in the art. Non-limiting examples of communicatively coupling may comprise Wi-Fi, Bluetooth, wired connections, wireless connection, quantum, and/or magnets. For ease of reference, the exemplary embodiment described herein refers to Wi-Fi and/or Bluetooth, but this description should not be interpreted as exclusionary of other electrical coupling mechanisms.

As used herein, the term “physical patient representation” refers to any model of the human body (e.g., as used in medical training and/or as an artist's lay figure) at any stage and/or age in development known in the art. Non-limiting examples of the physical patient representation may comprise a static manikin (hereinafter “manikin”), mannequin, and/or figure. For ease of reference, the exemplary embodiment described herein refers to a manikin and/or manikin, but this description should not be interpreted as exclusionary of other models of the human body.

As used herein, the term “virtual patient representation” refers to any graphical model of the human body (e.g., as used in medical training and/or as an artist's lay figure) at any stage and/or age in development known in the art. Non-limiting examples of the virtual patient representations may comprise a scan of a human and/or a model of a human body, a three-dimensional graphical interpretation of the human body, a two-dimensional graphical interpretation of a human body, a solid model of the human body, a wire-frame of the human body, and/or a surface model of the human body. For ease of reference, the exemplary embodiment described herein refers to a scan of a human and/or a model of a human body, but this description should not be interpreted as exclusionary of other graphical models of the human body.

The terms “about,” “approximately,” or “roughly” as used herein refer to being within an acceptable error range for the particular value as determined by one of ordinary skill in the art, which will depend in part on how the value is measured or determined, i.e., the limitations of the measurement system, i.e., the degree of precision required for a particular purpose, such real-time pricing of an activity and/or hotel. As used herein, “about,” “approximately,” or “roughly” refer to within ±15% of the numerical.

All numerical designations, including ranges, are approximations which are varied up or down by increments of 1.0, 0.1, 0.01 or 0.001 as appropriate. It is to be understood, even if it is not always explicitly stated, that all numerical designations are preceded by the term “about”. It is also to be understood, even if it is not always explicitly stated, that the compounds and structures described herein are merely exemplary and that equivalents of such are known in the art and can be substituted for the compounds and structures explicitly stated herein.

Wherever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “no more than,” “less than,” or “less than or equal to” precedes the first numerical value in a series of two or more numerical values, the term “no more than,” “less than” or “less than or equal to” applies to each of the numerical values in that series of numerical values. For example, less than or equal to 1, 2, or 3 is equivalent to less than or equal to 1, less than or equal to 2, or less than or equal to 3.

### Patient Examination System

The present disclosure pertains to a system and method for enhancing a physical patient representation through augmented reality (AR), virtual reality (VR), mixed reality (MR), and/or extended reality (XR) to augment the appearance of the physical patient representation and/or to simulate physical movements, aspects, and/or behaviors that the physical patient representation may be not capable of on its own, allowing for realistic movement and/or a diversity of races/ethnicities and/or disorders while still providing physical contact. In an embodiment the patient examination system may comprise a physical patient representation configured to be scanned such that a virtual patient representation may be displayed, via any display device known in the art (e.g., a video see-through head-mounted display (VST-HMD) and/or similar augmented reality (AR), virtual reality (VR), and/or mixed reality (MR) head-mounted displays).

As such, in this embodiment, the patient examination system may be configured to augment the appearance of the physical patient representation and/or to simulate physical movements, aspects, and/or behaviors that the physical patient representation (e.g., manikin) may not be capable of on its own. Furthermore, the patient examination system may allow at least one user an ability to touch the physical patient representation (e.g., manikin), such that an appropriate feedback response (e.g., touching an infant's appendage and the infant recoiling). As such, the patient examination system may optimize learning, training, mentoring, practice, case planning, and/or any assessment, evaluation, and/or research known in the art within a healthcare domain. Furthermore, the patient examination system may provide additional benefits for other domains, including but not limited to biology education, clothing retail, and/or any service and/or activity known in the art involving human representations.

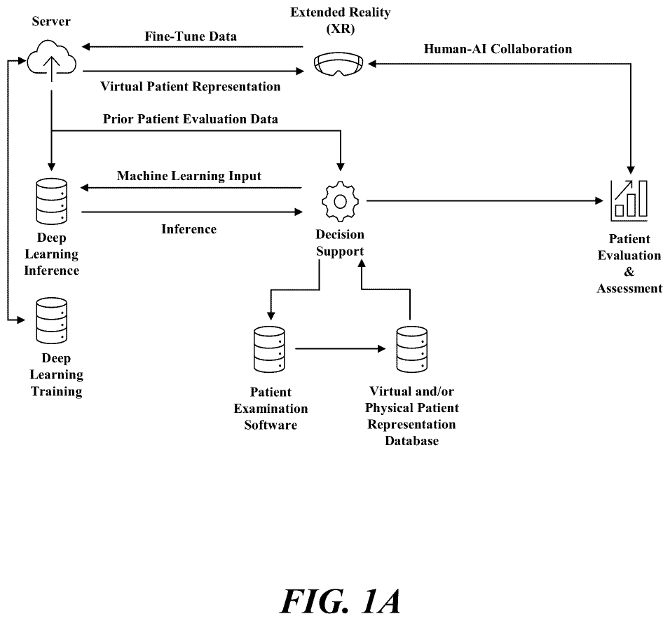

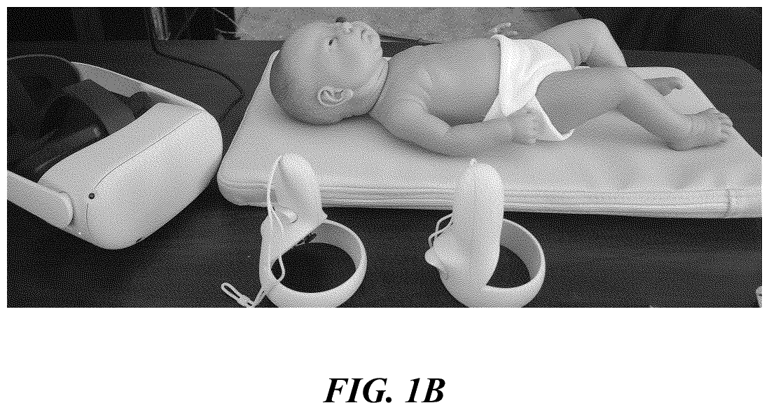

As shown in FIGS. 1A-1B, in an embodiment, the patient examination system may comprise a computing device comprising at least one processor, such that the at least one processor of the patient examination system may be communicatively coupled to at least one augmented reality (hereinafter “AR”) component (e.g., a Meta® Quest® 2 system) and/or virtual reality (hereinafter “VR”) component. The AR and/or VR component (hereinafter extended reality (“XR”) component) may comprise any AR and/or VR component known in the art.

In addition, the patient examination system may comprise AI-assisted patient evaluations and/or assessments, via the XR component, by employing state-of-the-art methods and/or algorithms from interdisciplinary practices, an overview of which is depicted in FIG. 1A. Machine learning is vastly used for robust and/or real-time detection of at least one health condition (e.g., cancer, ulcer, skin distortion, and/or skin discoloration) within and/or disposed upon a patient, whereas human-computer interaction concepts are employed for improving the assessment performance by including the judgement and/or training of the at least one user. As such, as shown in FIG. 1A, the patient examination system may employ a human-artificial intelligence collaboration via a headset to perform evaluations and/or analyses of the physical patient representation. As such, the patient examination system, via the XR component, may be configured to automatically detect and/or segment the defect regions using real-time deep learning operations instead of manually marking the defect regions in the XR environment within the XR component. In this way, the patient examination system optimizes the efficiencies related to improved assessment, evaluation, and/or testing of a patient by the at least one user, by highlighting and/or providing real-time reactions based on an inputted force by the at least one user.

Accordingly, as shown in FIG. 1A, the XR component of the patient examination system may comprise a deep-learning module residing within a memory of the computing device and/or on a server communicatively coupled to the computing device and/or at least one third-party database communicatively coupled to the computing device. The deep learning components of the patient examination system will be discussed in greater detail in the sections below; however, in use, the patient examination system may be configured to leverage real-time decision making by the at least one user with back-end AI-based recommendations resulting from at least one trained deep learning data set (e.g., a trained appropriate response data set and/or a trained known response data set with respect to an applied force). As such, the patient examination system may be particularly useful in a patient-based evaluation and/or training, in which routine assessments must take place to ensure the integrity and/or accuracy of a given patient examination, such as for appropriate diagnosis of ulcers, cuts, lacerations, broken bones, ulcers, diabetes, and/or cancer. FIG. 1B depicts a visual representation of the proposed methodology in use, according to an embodiment of the present disclosure.

Additionally, as shown in FIGS. 1A-1B, in this embodiment, the virtual and/or augmented reality system may be configured to augment a patient (e.g., a physical newborn), such that the patient examination system may provide a plurality of patient options to the at least one user (e.g., a range of children from infant to adolescent, a range of adults from an average adult to a geriatric patient, and/or a range of patients comprising a health condition, such as a diabetes, cancer, ulcers, cuts, and/or lacerations).

Moreover, as shown in FIGS. 1A-1B, in this embodiment, the patient examination system may comprise at least one display device (e.g., a AR and/or VR headset) associated with the computing device, such that the XR component of the patient examination system may be configured to transmit an electrical signal to the at least one processor, such that the display device may provide video see-through capabilities provided by at least one external camera disposed about at least a portion of the display device of the XR component. Furthermore, in this embodiment, the patent examination system may comprise at least one user-input actuator (e.g., a handheld controller) communicatively coupled to the at least one processor, such that an input by the at least one user may be tracked, recorded, and/or displayed within a 3D space on the display device associated with the computing device. As such, the at least one user-input actuator may be handheld. In addition, the patient examination system may also be configured to track an appendage (e.g., an arm and/or a hand) of the at least one user when the appendage of the at least one user may be placed in front of the display device, via the XR component.

In an embodiment, the physical representation may also comprise at least one sensor communicatively coupled to the at least one processor. As such, when the at least one user interacts with the physical patient representation, the at least one processor of the patient examination system may be configured to transmit an electric signal corresponding to the interaction to the XR component and/or the display device of the patient examination system. In this manner, the patient examination system may leverage the real-time XR component to allow for collaboration between at least one user and an AI platform within a physical patient representation itself, without relying on manual and/or inputted measurements from the at least one user to reconstruct the physical patient representation within the XR component.

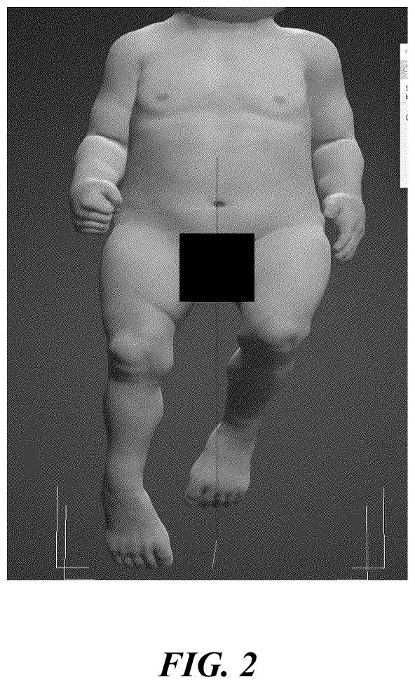

FIG. 2 depicts a graphical representation an exemplary configuration of a virtual model of a patient for a patient examination system, according to an embodiment of the present disclosure. As shown in FIG. 2, by using the video see-through capabilities of the XR component, the patient examination system may be configured to alter the appearance of the physical patient representation to represent any visual property (e.g., age, ethnicity, age), characteristic (e.g., build, male and/or female), and/or health condition (e.g., diabetes, cancer, skin discoloration, ulcers, cuts, and/or lacerations). As such, in an embodiment, the patient examination system, via the XR component, may be configured to overlay a graphical representation of a 3D virtual patient on top of the see-through video on the display device associated with the computing device in the same 3D space as the physical patient representation. Moreover, in this embodiment, the visual appearance of the graphical overlay of the virtual patient may supersede the visual appearance of the physical patient representation.

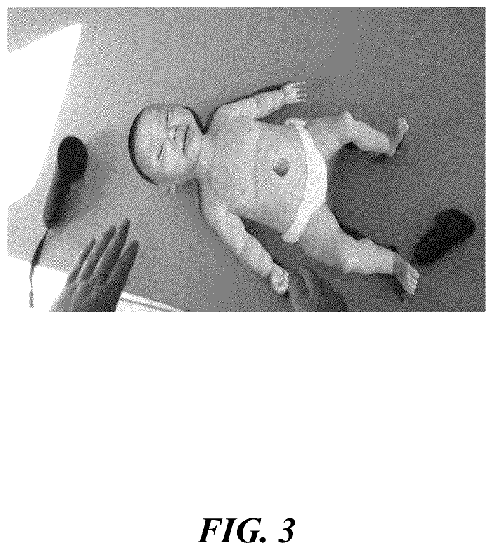

As shown in FIG. 3, for example, in an embodiment, while the physical patient representation may represent a Caucasian patient, the patient examination system may be configured to overlay an Asian virtual patient, such that the Asian virtual patient may be visualized in place of the physical patient representation within the display device, affording the perception of interacting with the Asian patient instead of the Caucasian patient.

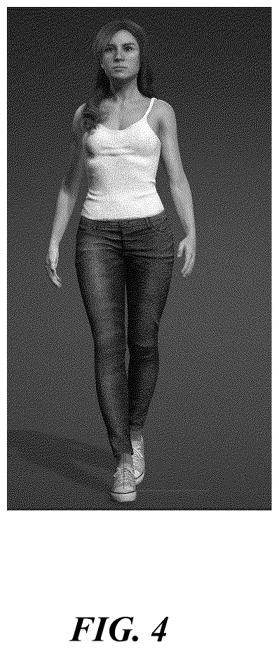

Additionally, as shown in FIG. 4, in another example, in an embodiment, while the physical patient representation may represent a young male patient (e.g., a male infant and/or a male child), the patient examination system may be configured to overlay a young female patient and/or an adult male patient and/or an adult female virtual patient (e.g., an adult male and/or female patient comprising a health condition and/or a geriatric male and/or female comprising a health condition), and vice versa. As such, via the patient examination system, the older female virtual patient may be visualized in place of the physical patient representation within the display device, affording the perception of interacting with the older female patient instead of the young male patient, and vice versa. In this manner, when the at least one user applies a force to the physical patient representation, via contact with the physical patient representation and/or the at least one user-input actuator, the at least one processor of the patient examination system may be configured to display the same applied force to the virtual patient representation overlayed with the physical patient representation.

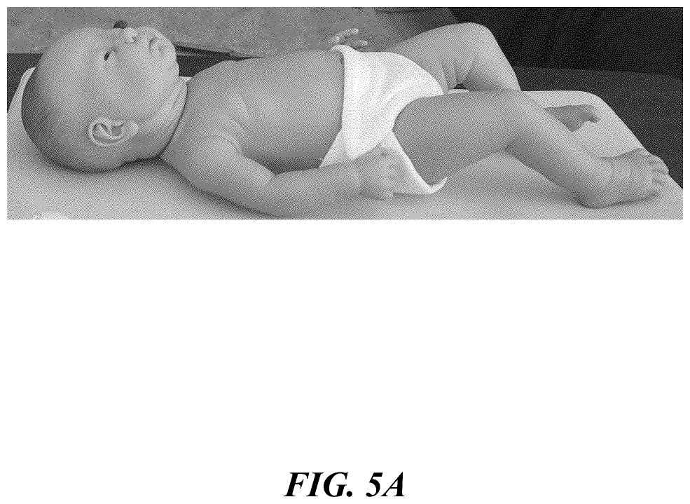

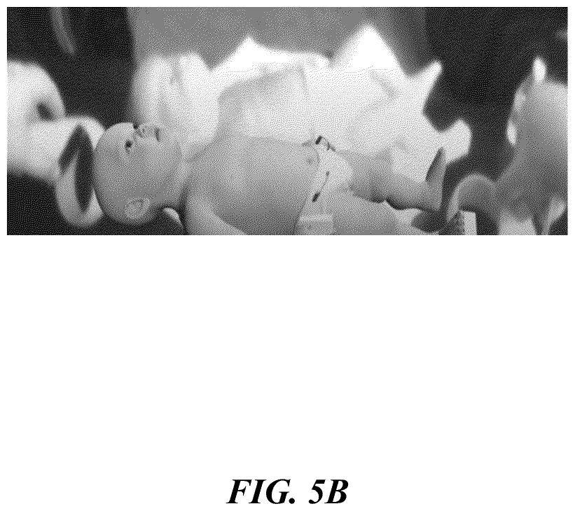

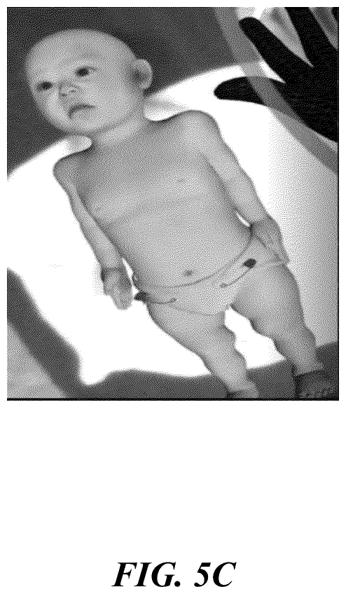

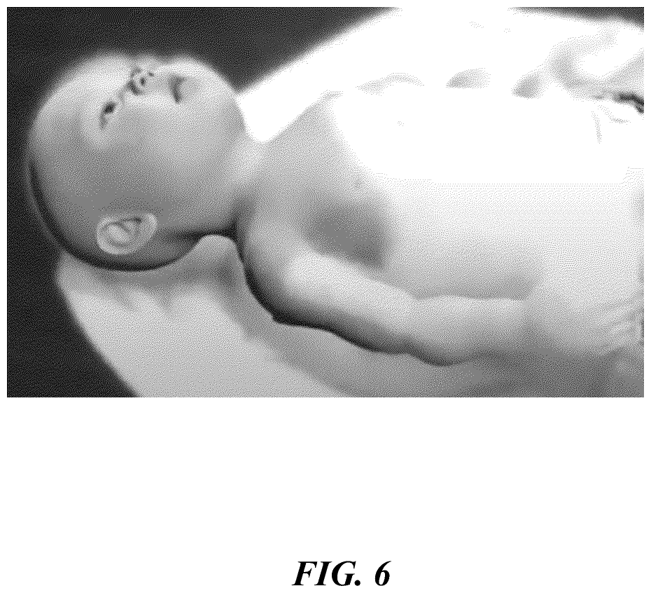

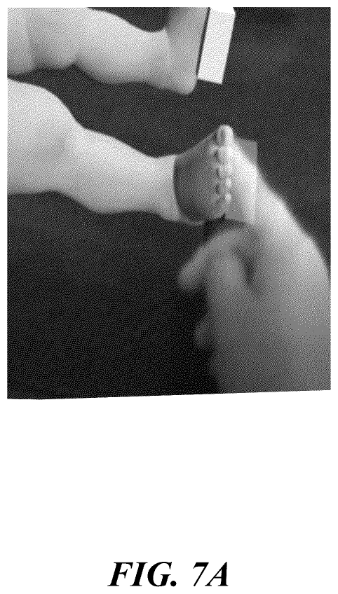

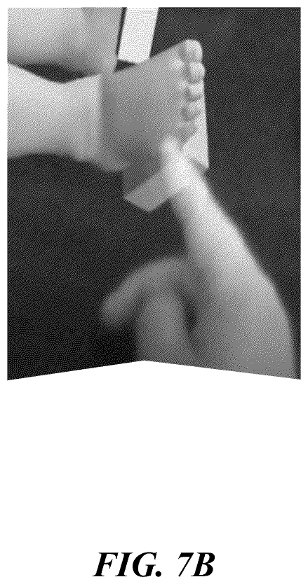

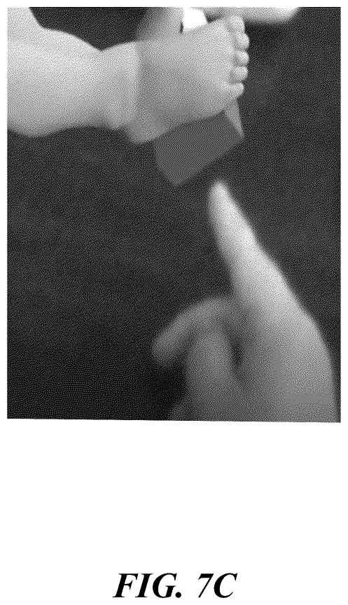

Additionally, as shown in FIGS. 5A-5C, in an embodiment, the patient examination system may be configured to alter and/or update the appearance and/or colorization of the virtual patient to represent a plurality of medical issues not afforded by the physical patient representation. Nonlimiting examples of the medical issues may comprise yellowing the skin and/or eyes to convey jaundice, using a pale and/or bluish virtual skin tone to convey cyanosis, and/or using a virtual skin texture with blemishes to convey acne. Moreover, as shown in FIGS. 6-7C, the patient examination system may be configured to implement plurality movements and/or behaviors of the virtual patient within the XR component used to visually represent actions that the physical patient representation is not capable of emulating. Non-limiting examples of the movements and/or behaviors may comprise an appendage of the virtual patient may be configured to convey a reflex response based on an interaction between the user and the virtual patient.

In an embodiment, as stated above, the patient examination system may comprise a deep-learning module, the deep-learning module comprising at least one trained appropriate response data set and/or trained known response data set and/or database within a memory of the computing device. The at least one appropriate and/or known response database comprising a plurality of appropriate and/or known responses by a human in response to a stimulus (e.g., reflex testing). In addition, in this embodiment, the patient examination system may comprise at least one Application Programming Interface (hereinafter “API”), such that the patient examination system may be configured to input at least one third-party database comprising a plurality of appropriate and/or known human responses to stimuli. Accordingly, as shown in FIGS. 7A-7C, in an embodiment, when the user interacts with the foot of the patient, the patient examination system may be configured to transmit a signal to the at least one processor, such that, within the XR component, the virtual patient may convey an appropriate and/or a known response based on the virtual patient. In an additional example, within the XR component of the patient examination system, in response to a stimulus, the head of the virtual patient may be slightly rotated, and/or the virtual mouth and/or eyes may be opened to convey at least one response reflex (e.g., a newborn attempting to root when the corner of the baby's mouth touches skin). Further, in another example, within the XR component of the patient examination system, the at least one processor may be configured to transmit at least one electrical signal, such that the display device shows the virtual patient closing its virtual fingers to virtually grasp the user's finger when the finger touches the physical representation's palm (e.g., the palmar grasp reflex). As such, other human reflexes may also be simulated, via the patient examination system.

Moreover, in an embodiment, the deep-learning module of the patient examination system may comprise a plurality of trained background data sets and/or a plurality of trained health information data sets with respect to the virtual patient representation. In this manner, in this embodiment, the patient examination system may be configured to transmit an electrical signal to the display device, such that at least one of the plurality of trained background data sets and/or at least one of the plurality of trained health information data sets may be displayed within the XR environment, such that the display device overlays the view of the at least one user with the at least one of the plurality of trained background data sets and/or at least one of the plurality of health information data sets.

In addition, in an embodiment, the deep-learning module of the patient examination system and/or the computing device of the patient examination system may be communicatively coupled to at least one alternative computing device and/or at least one alternative display device. As such, via the patient examination system, at least one alternative user (e.g., a patient training administrator) may view, in real-time, the interaction of the at least one user and the virtual patient representation. Accordingly, the patient examination system may be configured to transmit and/or display the view of the at least one user within the XR environment to the at least one alternative computing device and/or display device, in real-time, such that the at least one alternative user (e.g., the exam administrator) may also view the perspective of the at least one user. In this manner, the patient examination system may be configured to display at least one of the plurality of trained background data sets and/or at least one of the plurality of trained health information data sets within the at least one alternative display device, such that the at least one alternative user may monitor the progress and/or examination by the at least one user, in real-time.

In an embodiment, the deep-learning module of the patient examination system may also comprise a plurality of trained pre-recorded statements, questions, and/or responses data sets, such that the virtual patient representation may provide at least one prompt from the plurality of prerecorded statements, questions, and/or responses. As such, in this embodiment, based on the at least one trained pre-recorded statements, questions, and/or responses, when the at least one user interacts with the physical patient representation (e.g., using the at least one user-input actuator, and/or transferring a force upon the physical patient representation), the deep-learning module may be configured to provide at least one additional response with respect to the interaction of the at least one user. In addition, in this embodiment, the at least one processor of the patient examination system may be configured to record the statements made by the at least one user during the examination, via at least one microphone, within a memory of the computing. Accordingly, the patient examination system may be configured to retrain at least one of the pluralities of trained pre-recorded statements, questions, and/or responses data sets, with the at least one recorded statement from the at least one user.

In an embodiment, the deep-learning module of the patient examination system may also comprise at least one trained movement and/or sound data set and/or database within the memory of the computing device. As such, the at least one movement and/or sound database of the patient examination system may comprise a plurality of human movements, via animations, patient sounds, via recorded and/or synthesized audio, and/or symbolic data, such as the virtual patient's medical history, as shown in FIG. 5. As shown in FIGS. 5-8C, the physical patient representation may comprise any patient representation known in the art. Non-limiting examples of the patient representation may comprise a representation as simple as a static mannikin and/or figure and/or as complex as a full-body patient representation with simulation capabilities like breathing sounds and heart rate. As shown in FIG. 6, in conjunction with FIGS. 7A-7C, in an embodiment, the patient examination system may be provided within the XR component a physical representation comprising passive haptics (e.g., the perception of physically touching and grasping the virtual patient representation due to touching and grasping the physical patient representation while visually seeing one's hands touching and grasping the virtual patient) within the display device. As such, in an embodiment, the at least one processor of the patient examination system may be communicatively coupled to the physical patient representation. In this manner, the physical patient representation may comprise any electrical and/or mechanical patient representation known in the art. Furthermore, any features of the physical patient representation known in the art (e.g., breathing sounds) may also be leveraged by patient examination system, via the at least one movement and/or sound database and/or at least one third-party database, via the API. For example, in some embodiments, the patient examination system may be configured to output breathing sounds of the physical patient representation (e.g., manikin) while simultaneously displaying the virtual patient representation in the XR component environment and/or data on the display device associated within the computing device.

Moreover, as shown in FIG. 3, in conjunction with FIG. 4 and FIGS. 7A-7C, in an embodiment, the patient examination system may be configured to display and/or output the augmented and/or virtual patient representation as a virtual patient within a virtual environment. In this manner, the patient examination system, via the display device associated with the XR component, may be configured to replace the user's view of the physical patient representation with at least one computer-generated view of the virtual patient. In this embodiment, the at least one processor may be configured to transmit a signal to the display device, such that at least one pixel in the see-through video of the physical representation may be replaced with at least one pixel of the computer-generated view, providing a virtual patient representation. As such, while a current implementation with AR and/or VR may be limited to providing black-and-white see-through capabilities of the real world, the patient examination system may overlay the XR component of the real world with a colored and/or computer-generated image of the virtual patient representation.

Furthermore, in an embodiment, as shown in FIGS. 2-3, in conjunction FIGS. 7A-7C, the virtual patient representation may comprise spatial tracking capabilities of the XR component. In this manner, the patient examination system may be configured to enable the perspective of the virtual patient to be automatically updated, in real-time, relative to the perspective of the physical patient representation (e.g., manikin). In this manner, the spatial tracking capabilities of the patient examination system may comprise at least one static calibration of the physical space and/or at least one dynamic tracking feature, via at least one sensor communicatively coupled to the at least one processor, such that at least one physical space (e.g., an examination room) around the physical patient representation (e.g., a physical simulator; manikin) may be tracked. In addition, the patient examination system may be configured to use the at least one user-input actuator as at least one anchor within the XR component to register the position, orientation, and/or scale of the physical patient representation, such that the corresponding image may be displayed on the display device, via the at least one processor. For example, in some embodiments, the patient examination system may determine the position, orientation, and/or scale of the physical patient representation by placing at least one user-input actuator at the feet of the physical patient representation and the other user-input actuator at the head of the physical representation.

As shown in FIG. 6, in conjunction with FIG. 3, and FIGS. 7A-7C, the patient examination system may be configured to detect where the hands of the at least one user are relative to the physical and/or virtual patient representations, via the spatial tracking capabilities of the virtual patient representation within the XR component. As such, as shown in FIG. 5, via the spatial tracking, the patient examination system may also allow several interactive possibilities between the at least one user and the virtual patient representation. Non-limiting examples of the interactive possibilities may comprise viewing virtual gloved hand representations of their bare physical hands and/or using a pass-through filter to show the user their physical hands from the see-through video occluding the virtual patient representation when they rest their physical hands-on top of the chest of the physical patient representation, as shown in FIGS. 6-7C.

In an embodiment, as shown in FIGS. 7A-7C, in conjunction with FIGS. 2-3, the patient examination system may be configured to supersede the visual characteristics of the physical patient representation with the visual characteristics of the virtual patient representation (e.g., jaundice, cyanosis, acrocyanosis, and other skin changes, such as newborn acne) within the display device, via the video see-through capabilities of the XR component. Additionally, in this embodiment, the patient examination system may also be configured to supersede the visual behaviors of the physical patient representation with the visual behaviors of the virtual patient representation (e.g., blinking, opening the mouth) within the display device, via the video see-through capabilities of the XR component. Furthermore, the at least one user may have the ability to physically touch, grasp, and/or handle the virtual patient representation by leveraging the passive haptics of the physical and/or virtual physical representation, via the at least one sensor, the co-located physical patient representation, and/or the hand tracking capabilities of the XR component of the patient examination system.

Additionally, in an embodiment, the patient examination system may also be configured allow the user to physically interact with the virtual patient representation using physical tools (e.g., a measuring tape). In this embodiment, the patient examination system may input at least one passive haptic input afforded by the physical patient representation. Moreover, in this embodiment, the patient examination system may input at least one computer vision-based approach within the memory of the computing device and/or at least one third-party database, such that the patient examination system may identify the at least one pixel within the see-through video that correspond to the physical tool (e.g., the color of the measuring tape). As such, at least one computer vision-based approach may be used to occlude the virtual patient representation (e.g., the physical measuring tape can be seen in the see-through video laying on top of the virtual patient's chest). Furthermore, in an embodiment, at least one additional virtual representation (e.g., a head-mounted display) may be configured to be implemented into the patient examination system, including, but not limited to advanced XR component (e.g., the Meta® Quest® Pro 2) and/or a colored see-through video, as opposed to black-and-white see-through video. As such, in this embodiment, the patient examination system may be configured to develop a plurality of virtual patients and/or scenarios, via the at least one processor of the computing device and/or the XR component (e.g., Unity, Unreal Engine).

In an embodiment, the patient examination system may be configured to compare and/or evaluate the at least one response generated via the at least one inputted stimulus from the at least one user with at least one of the pluralities of trained appropriate responses, known responses, movements, and/or sounds data sets, via at least one patient examination algorithm. In this manner, the patient examination may be configured to automatically provide an examination score of the at least one user, in real-time. As such, in this embodiment, the patient examination system may comprise at least API, such that the patient examination system may communicatively couple to at least one third-party database comprising a plurality of additional trained appropriate responses, known responses, movements, and/or sounds data sets. Accordingly, in this embodiment, the patient examination system may be configured to input the at least one generated response, movement, and/or sound based on the at least one stimulus inputted, via the at least one user-input actuator within the plurality of trained appropriate responses, known responses, movements, and/or sounds data sets, such that, via at least one deep-learning algorithm, the patient examination system may be configured to updated and/or retrain the plurality of trained appropriate responses, known responses, movements and/or sounds data sets, optimizing patient examination training, via the patient examination system, for each virtual examination.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

### INCORPORATION BY REFERENCE

Welch, Gregory et. al, Physical-Virtual Patient Bed System. U.S. Pat. No. 9,679,500 B2, United States Patent and Trademark Office, 13 Jun. 2017.
Brooke J. SUS: a “quick and dirty” usability scale. In: Jordan P W, Thomas B, Werdmeester B A, McClelland I L, eds., Usability Evaluation in Industry. London: Taylor & Francis; 1996:189-194.
Kim H K, Park J, Choi Y, Choe M. (2018). Virtual reality sickness questionnaire (VRSQ): motion sickness measurement index in a virtual reality environment. Appl Ergon 2018;69:66-73. doi:10.1016/j.apergo.2017.12.016
Leighton K, Ravert P, Mudra V, Macintosh C. Updating the Simulation Effectiveness Tool: item modifications and reevaluation of psychometric properties. Nurs Educ Perspect 2015;36(5):317-323.
Leighton K, Ravert P, Mudra V, Macintosh C. Simulation Effective Tool Modified Virtual. Available at: https://sites.google.com/view/evaluatinghealthcaresimulation. Accessed Jan. 9, 2023.
U.S. General Services Administration. System usability scale. Usability.gov. Available at: https://www.usability.gov/how-to-and-tools/methods/system-usability-scale.html. Accessed May 31, 2023.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. A patient examination system for optimizing a physical patient examination, the system comprising:
an extended reality component communicatively coupled to at least one user-input actuator, the extended reality component configured to scan at least one portion of a physical patient representation to overlay a virtual patient representation on the physical patient representation, the user-input actuator configured to receive at least one stimulus from a user to at least one portion of the overlayed virtual patient representation;
a computing device having at least one processor communicatively coupled to the extended reality component, the computing device configured to receive the scan of the at least one portion of the physical patient representation from the extended reality component;
wherein the computing device is communicatively coupled to a display device, the display device configured to visualize at least one portion of the virtual patient representation; and
wherein upon receiving the stimulus from the at least one user, the extended reality headset generates a response within the overlayed virtual patient representation disposed upon at least one portion of the physical patient representation, whereby the extended reality component transmits the response to the display device.

2. The patient examination system of claim 1, wherein a memory of the computing device comprises a deep-learning module comprising a plurality of trained appropriate responses, trained known responses, or both.

3. The patient examination system of claim 2, wherein when the at least one user provides a stimulus to the virtual reality representation, the extended reality component is configured to transmit a signal to the at least one processor, whereby the virtual patient representation conveys the at least one trained appropriate response, at least one trained known response, or both based on the provided stimulus.

4. The patient examination system of claim 2, wherein the deep leaning module further comprises a plurality of trained movements, trained sounds, or both.

5. The patient examination system of claim 1, wherein the at least one processor is configured to alter at least one visual characteristic of the physical patient representation with at least one visual characteristic of the virtual patient representation within the display device associated with the computing device, the extended reality component, or both.

6. The patient examination system of claim 2, wherein the deep-learning module is communicatively coupled to at least one alternative computing device, at least one alternative display device, or both.

7. The patient examination system of claim 6, wherein the at least one processor is configured to display an interaction of the at least one user and the virtual patient representation on the at least one alternative computing device, at least one display device or both, whereby at least one alternative user views, in real-time, the interaction of the at least one user and the virtual patient representation.

8. The patient examination system of claim 7, wherein the deep-learning module further comprises a plurality of trained background data sets, a plurality of trained health information data sets, or both with respect to the virtual patient representation.

9. The patient examination system of claim 8, wherein the at least one processor is configured to overlay at least one of the plurality of trained background data sets, at least one of the plurality of trained health information data sets, or both of the virtual patient representation with the view of an interaction of the at least one user and the virtual patient representation on the display device, simultaneously and in real-time.

10. The patient examination system of claim 1, wherein when the extended reality component overlays at least one portion of the physical patient representation with at least one associated potion of the virtual patient representation, the extended reality component replaces at least one aspect of the physical patient representation with at least one computer-generated aspect of the virtual patient representation within the display device associated with the computing device, the extended reality component, or both.

11. A method for optimizing patient examination training, the method comprising:
scanning a physical patient representation disposed about an extended reality component, wherein a virtual patient representation is overlayed upon at least one portion of the scanned physical patient representation;
generating, via the extended reality component, a response associated with an inputted stimulus from at least one user onto at least one portion of the virtual patient representation, wherein the stimulus is inputted via at least one user-input actuator communicatively coupled with the extended reality component;
comparing, via a computing device having at least one processor communicatively coupled to the extended reality component, the associated response with a plurality of trained appropriate responses, trained known responses, or both; and
transmitting, via the computing device, an examination score to a display device associated with the computing device, the extended reality component, or both, wherein the examination score is calculated based on the comparison between the associated response and at least one response of the plurality of trained appropriate responses, trained known responses or both.

12. The method of claim 11, wherein a memory of the computing device comprises a deep-learning module comprising a plurality of trained appropriate responses, trained known responses, or both.

13. The method of claim 12, further comprising the step of, transmitting, via the extended reality component, at least one signal to the at least one processor, wherein the virtual patient representation conveys the at least one appropriate response, at least one known response, or both based on the provided stimulus.

14. The method of claim 12, wherein the deep leaning module further comprises a plurality of trained movements, trained sounds, or both.

15. The method of claim 11, further comprising the step of, altering, via the at least one user-input actuator, at least one visual characteristic of the physical patient representation with at least one visual characteristic of the virtual patient representation within the display device associated with the computing device, the extended reality component, or both.

16. The method of claim 12, wherein the deep-learning module is communicatively coupled to at least one alternative computing device, at least one alternative display device, or both.

17. The method of claim 16, further comprising the step of, displaying, via the at least one processor, an interaction of the at least one user and the virtual patient representation on the at least one alternative computing device, at least one alternative display device, or both, wherein at least one alternative user views, in real-time, the interaction of the at least one user and the virtual patient representation.

18. The method of claim 16, wherein the deep-learning module further comprises a plurality of trained background data sets, a plurality of trained health information data sets, or both with respect to the virtual patient representation.

19. The method of claim 18, further comprising the step of, overlaying, via the at least one processor, at least one of the plurality of trained background data sets, at least one of the plurality of trained health information data sets, or both of the virtual patient representation with the view of an interaction of the at least one user and the virtual patient representation on the display device, simultaneously and in real-time.

20. The method of claim 11, further comprising the step of, replacing at least one aspect of the physical patient representation with at least one computer-generated aspect of the virtual patient representation within the display device associated with the computing device, the extended reality component, or both.
